(19) United States
(12) Patent Application Publication
(10) Pub. No.: US 2010/0267143 A1
(43) Pub. Date: Oct. 21, 2010

(54) Method for Surface Modification of Polymeric Scaffold for Stem Cell Transplantation Using Decellularized Extracellular Matrix

(75) Inventors: Kwi Deok Park, Seoul (KR); Hee Joong Kim, Seoul (KR); Dong Keun Han, Seoul (KR); Yu Jin Hong, Seoul (KR); Heung Jae Chun, Seoul (KR); Ju Woong Jang, Seoul (KR)

Correspondence Address: DRINKER BIDDLE & REATH;ATTN: INTELLECTUAL PROPERTY GROUP, ONE LOGAN SQUARE, SUITE 2000, PHILADELPHIA, PA, 19103-6996, US

(73) Assignee: SNU R&DB Foundation, Seoul (KR)

(21) Appl. No.: 12/607,793

(22) Filed: Oct. 28, 2009

(30) Foreign Application Priority Data
Apr. 16, 2009 (KR) 10-2009-0033079
Oct. 20, 2009 (KR) 10-2009-0099579

(51) Int. Cl. C12N 5/071

(52) U.S. Cl. 435/402

(57) ABSTRACT

The present invention relates to a method for the surface modification of a polymeric scaffold for stem cell transplantation using a decellularized extracellular matrix. The method for the surface modification of the polymeric scaffold according to the present invention can embody a biomimetic surface environment that is effective for initial cell attachment, cell growth and differentiation of stem cells by modifying the surface of the polymeric scaffold using the decellularized extracellular matrix directly derived from specific tissue cells.

### CROSS REFERENCE TO RELATED APPLICATIONS

This application claims priority under 35 U.S.C. §119(a) to Korean Application No. 10-2009-0033079, filed on Apr. 16, 2009, and Korean Application No. 10-2009-0099579, filed Oct. 20, 2009, each of which application is incorporated by reference herein in its entirety.

### BACKGROUND OF THE INVENTION

Technology related to fundamental and applied tissue engineering has been advanced for the purpose of developing transplantable artificial tissues as part of regenerative medicine. Specifically, studies including stem cell proliferation and differentiation, development of cytocompatible and biocompatible three-dimensional scaffolds, and construction of a variety of tissue engineering tools are now the most active research areas in regenerative medicine. Among them, three-dimensional scaffolds that are used to deliver stem cells or tissue cells therein are critical for the development of artificial tissues and organs.

Scaffold materials used for the regeneration of body tissues must act as a platform to which cells adhere to form three-dimensional tissues. They must also function as a temporary barrier between transplanted cells and host cells, and they must be nontoxic and biocompatible generating tolerable immune reactions, if any are to be generated. In addition, scaffold material must be biodegradable in vivo at a desired time when the transplanted cells have grown sufficiently to the point of being able to adequately function as a tissue.

Typically, scaffolds are prepared from synthetic or natural polymers or their composites, and are manufactured into three-dimensional structures which have a variety of morphologies and properties. Most commonly used synthetic biodegradable polymers include polyglycolic acid (PGA), polylactic acid (PLA), poly(lactic acid-co-glycolic acid) (PLGA), poly-ε-caprolactone (PCL), and derivatives and copolymers thereof, which can be used as biomaterials for scaffold preparation. Naturally biodegradable polymers as exemplified by collagen, alginate, hyaluronic acid, gelatin, chitosan, fibrin, etc., are also very useful candidates for this purpose. A variety of different forms of materials, such as sponges, gels, fibers, and microbeads, are applied for the fabrication of scaffolds, and the most popular ones are porous sponges and injectable hydrogels.

There are many technical barriers in achieving the goal of tissue engineering perfectly. The most critical one among these technical barriers is the development of a core technology with regard to the scaffold that can generate a cytocompatible and biomimetic surface environment within or onto the scaffold. In light of the fact that scaffolds are fundamental in providing a three-dimensional environment and should be advantageous for cell adhesion and growth, properties of the scaffold surface to which cells adhesion plays a critical role in determining the present behaviors and future fate of cells.

Polymer surface to which cells directly attach is one of the most important factors in scaffold design that requires careful and multiple considerations. On the whole, cell adhesion to natural polymers is much easier to accomplish than that to synthetic polymers, and techniques used are typically selected from 1) surface coating or grafting of scaffolds with materials which cell surface receptors can recognize, such as fibronectin, an alginine-glycine-aspartic acid (RGD) peptide, vitronectin, laminin, etc., 2) treating a polymeric scaffold surface with a natural polymer, such as collagen, gelatin, fibrin, etc., and 3) preparing a hybrid scaffold from a mixture of synthetic and natural polymers. However, these approaches are only partly effective in ensuring cell adhesion and especially stem cell differentiation. In fact, current technologies of surface modifications have limitations in creating a biomimetic surface environment that the cells recognize as being natural and autogenous.

Extracellular matrix (ECM) is a physical entity in the extracellular domain that is composed mainly of a cell-derived network structure of fibrous proteins, polysaccharides, and other minor constituents. Its components include structural elements such as collagen and elastin, and adhesive proteins such as fibronectin, laminin, vitronectin, and tenascins, as well as proteoglycans such as chondroitin sulfate and heparan sulfate, and non-proteoglycans. The hierarchical network of these ECM components provides a natural environment in which cells can survive and function properly. In fact, cell-ECM interactions are essential in maintaining tissue homeostasis and in regulating cell proliferation and differentiation.

Therefore, many studies have been carried out, utilizing ECM as a valuable resource for tissue engineering and regenerative medicine. For example, living tissues such as small intestine submucosa (SIS), bladder, and skin, are directly decellularized to obtain a cell-free matrix and then they are used for cell transplantation. In addition, Korean Patent No. 10-715505 describes a method of preparing a cell-derived ECM scaffold. In this case, the chondrocyte/ECM membrane was centrifuged, then cultured into a pellet-type structure for a period of time, followed by freeze-drying to produce a porous scaffold, which is supposed to be applied for cartilage regeneration.

There exists in the art a need for an ECM-based scaffold that is built for a biomimetic environment that is useful for stem cell transplantation and tissue regeneration.

### SUMMARY OF THE INVENTION

Surface modification of scaffolds was intensively and thoroughly researched by the present inventors with the goal of providing a biomimetic surface environment. This research resulted in the finding that a surface modified with an extracellular matrix left behind after decellularization of the cells transplanted thereon allows cells to readily adhere thereto and also provides a biomimetic environment suitable for the growth and differentiation of stem cells.

An objective of the present invention is to provide a method for modifying the surface of a polymeric scaffold for stem cell transplantation using a decellularized extracellular matrix.

In accordance with the present invention, the above objective is accomplished by providing a method for the surface modification of polymeric scaffolds for stem cell transplantation. The method comprising: 1) mixing a solution of an organic solvent containing a biodegradable organic polymer dissolved therein with an inorganic ingredient or a natural polymer, followed by gas foaming of the mixture in the presence of NaHCO₃ and freeze-drying of the mixture to make a porous composite scaffold; 2) subjecting the porous composite scaffold to plasma treatment, sterilization, transplantation with tissue cells, and cell culture thereon and decellularization using a decellularization solution in that order; and 3) transplanting and culturing stem cells in the decellularized scaffold.

### BRIEF DESCRIPTION OF THE DRAWINGS

The above and other objects, features and other advantages of the present invention will be more clearly understood from the following detailed description taken in conjunction with the accompanying drawings, in which:

FIG. 1, comprising FIG. 1A-1F, is a series of images of fluorescence microphotographs showing the presence of cell nuclei and type 1 collagen on surface-modified polymeric scaffolds after DAPI(4′-6-diamidino-2-phenylindole) staining and immunofluorescent staining.

FIG. 2, comprising FIG. 2A-2I, is a series of images of Alizarin red staining showing the ability of the surface-modified polymeric scaffolds in inducing a mineralized matrix from osteogenic differentiation of stem cells.

### DETAILED DESCRIPTION OF THE INVENTION

The present invention relates to a method for modifying the surface of a polymeric scaffold for stem cell transplantation using a decellularized extracellular matrix.

The method for the surface modification of polymeric scaffolds for stem cell transplantation comprises: 1) mixing a solution of an organic solvent containing a biodegradable organic polymer dissolved therein with an inorganic ingredient or a natural polymer, followed by gas foaming of the mixture in the presence of NaHCO₃ and freeze-drying of the mixture to make a porous composite scaffold; 2) subjecting the porous composite scaffold to plasma treatment, sterilization, transplantation with tissue cells, and cell culture thereon and decellularization using a decellularization solution in that order; and 3) transplanting and culturing stem cells in the decellularized scaffold.

Hereinafter, a detailed description will be given of the present invention.

In the method of the present invention, in step 1) a porous composite scaffold is prepared. In greater detail, a biodegradable polymer is completely dissolved in an organic solvent and mixed with an inorganic component or a natural polymer. Meanwhile, sodium bicarbonate (NaHCO₃) is sieved through the meshes to have the particles ranging from 200 μm to 300 μm in size. Once these NaHCO₃ particles are blended with the mixed polymeric solution, the composite pastes are packed in a disc-shaped silicon mold, and rapidly frozen in the liquid nitrogen. After a predetermined time elapses, the frozen blend is taken out and lyophilized to completely remove the solvent. The disc-type matrix is then subjected to gas foaming in citric acid solution for 2 days, and washed with distilled water, then lyophilized again to yield a porous 3 dimensional composite scaffold.

Examples of the biodegradable polymers useful in the present invention include but are not limited to polyglycolic acid (PGA), polylactic acid (PLA), poly(lactic-co-glycolic acid) (PLGA), poly-ε-caprolactone (PCL), polyamino acid, derivatives, and copolymers thereof.

Non-limiting examples of the inorganic component include hydroxyapatite (HA), β-tricalcium phosphate (β-TCP), biphasic calcium phosphate (BCP), calcium carbonate and glass ceramics.

The natural polymers may be exemplified by collagen, a modified collagen, alginate, a modified alginate, hyaluronic acid, a modified hyaluronic acid, gelatin, a modified gelatin, chitosan, a modified chitosan, fibrin, a modified fibrin, and a combination thereof, but is not limited thereto.

The organic solvents may be selected from a group consisting of methylene chloride, hexane, chloroform, acetone, dioxane, tetrahydrofuran, hexafluoroisopropane and a combination thereof.

In the method of the present invention, in step 2) cells are transplanted onto and cultured in the composite scaffold. At a predetermined time, the composite scaffold is decellularized using a decellularization solution. In greater detail, the fabricated composite scaffold is hydrophilized by plasma treatment in order to facilitate cell adhesion thereonto, followed by sterilization. Tissue cells are then transplanted onto and cultured in the sterilized composite scaffold for a while and washed with phosphate buffered saline. Decellularization is performed using a decellularization solution to remove the cellular components other than those in the ECM.

Examples of tissue cells useful in the present invention include, but are not limited to, fibroblasts, chondrocytes, endothelial cells, smooth muscle cells, osteoblasts, hepatocytes, nerve cells, cardiomyocytes, intervertebral disc cells and a combination thereof. The tissue cells may be autologous, homologous, or heterologous cells.

The decellularization solution used in the present invention comprises at least one selected from a group consisting of lysis solution, hypotonic solution, cationic-surfactant, anionic-surfactant, nonionic-surfactant, RNase A, and DNase I. In particular, the decellularization solution comprises 10 mM Tris-HCl (pH 8.0), 1.5M NaCl, 1% EDTA, 1% Triton X-100, RNase A, and DNase I.

After the transplantation of tissue cells onto the sterilized scaffold, biologically active biomolecules may be added during the culture of the tissue cells to promote the proliferation of tissue cells or the secretion of ECM. The biologically active biomolecules may be selected from a group consisting of transforming growth factor-beta (TGF-β), fibroblast growth factor (FGF), bone morphogenetic protein (BMP), vascular endothelial growth factor (VEGF), epidermal growth factor (EGF), insulin-like growth factor (IGF), platelet-derived growth factor (PDGF), nerve growth factor (NGF), hepatocyte growth factor (HGF), placental growth factor (PIGF), granulocyte colony stimulating factor (G-CSF), medium supplements such as ascorbate 2-phosphate, and a combination thereof.

In the method of the present invention, in step 3) stem cells are transplanted onto the decellularized scaffold and cultured for a certain period of time.

The stem cells are autologous stem cells. Examples of the stem cells useful in the present invention include embryonic stem cells, bone marrow-derived stem cells, adipose-derived stem cells, placenta-derived stem cells, induced pluripotent stem cells and a combination thereof, but are not limited thereto.

The transplantation and culture of stem cells may be carried out in the presence of a biologically active biomolecules that can promote stem cell differentiation into specific lineages. The biologically active biomolecules of step 2) may be used again.

After the surface modifications using the decellularized extracellular matrix of a specific cell origin, the polymeric scaffold of the present invention provides a biomimetic environment which guarantees cell adhesion thereto and promotes the growth and differentiation of stem cells thereon. Therefore, the surface-modified polymeric scaffold of the present invention is useful in a wide range of applications with stem cells, targeting specific tissue regeneration of a variety of different tissues including cartilage, bone, nerves, myocardium, liver, and skin.

The following examples are intended to further illustrate the present invention. However examples and experimental examples are shown only for better understanding of the present invention without limiting its scope.

### EXAMPLES AND EXPERIMENTAL EXAMPLES

### Example 1

### Surface Modification of Polymeric Composite Scaffold [PLGA/HA/TCP-Decellularization/Fibroblast]

1. Preparation of a Porous Composite Scaffold

Poly(lactic acid-co-glycolic acid) (PLGA, 75:25, molecular weight-110,000; Boehringer Ingelheim, Ingelheim, Germany) was dissolved in methylene chloride to give a 13% (w/v) PLGA solution. To this was added a mixture of the inorganic components, hydroxyapatite (HA) and β-tricalcium phosphate (β-TCP) in such an amount as to maintain a weight ratio of 1:0.2:0.8 for PLGA: HA: β-TCP. Meanwhile, sodium bicarbonate (NaHCO₃) was sieved through the meshes to generate particles ranging from 200 μm to 300 μm in size. These particles were homogeneously blended with the mixture of PLGA solution and the inorganic components. The resulting blend was placed in a disc-shaped silicon mold, 8 mm in diameter and 2 mm in thickness, and rapidly frozen in liquid nitrogen. After a short period of time, the molded scaffold was taken out of the mold and subjected to lyophilization for 2 days to completely remove the solvent. Afterwards, the disc-type scaffolds were allowed to undergo gas foaming for 2 days in a 20% citric acid solution that was changed twice every day. Finally, they were washed with distilled water and lyophilized to generate a porous composite scaffold.

2. Surface Plasma Treatment of the Composite Scaffold

Hydrophilization was induced on the surface of the composite scaffold by using plasma treatment, in order to facilitate cell adhesion to the surface. For this, RFGD (radio frequency glow discharge; Model ATPT-1000, CLIOTEK, Inc., Seoul, Korea) was employed. The composite scaffold was placed in the chamber of the plasma apparatus and subjected to plasma treatment for 30 seconds in an argon gas atmosphere with the inner pressure set to 10⁻³ torr using a vacuum pump. The plasma-treated scaffold was stored at room temperature in a dehumidified condition.

3. Cell Transplantation and Culture in the Composite Scaffold

The plasma-treated composite scaffolds were placed in 24-well plates and transplanted with fibroblasts at a density of 5×10⁵ cells per scaffold in DMEM supplemented with 10% fetal bovine serum and 1% penicillin/streptomycin, followed by culturing and then replacing the medium twice a week with fresh medium.

4. Decellularization of the Cell-Transplanted Composite Scaffold

Following culture for one week, the scaffold was washed with phosphate buffered saline (PBS) and immersed in a decellularization solution [10 mM Tris-HCl (pH 8.0), 1.5M NaCl, 1% EDTA, 1% Triton X-100, RNase A, and DNase I]. The scaffolds were vortexed several times and then sealed, followed by incubation at 37° C. for 48 hrs in a water bath with shaking at 120 rpm. Then, the samples were taken out and briefly washed with PBS in a clean bench. They were again immersed in PBS and washed for 24 hrs. Finally, the samples were dried, sealed, and stored at −20° C.

5. Stem Cell Transplantation and Culture in the Decellularized Composite Scaffold

Stem cells isolated from rabbit bone marrow were transplanted at a density of 5×10⁵ cells per decellularized composite scaffold and cultured in an osteogenic medium for four weeks.

### Example 2

### Surface Modification of a Polymeric Composite Scaffold [PLGA/HA/TCP-Decellularization/Pre-osteoblast]

A decellularized composite scaffold was prepared in the same manner as in Example 1 with the exception that pre-osteoblasts were used instead of the fibroblasts of Example 1-3.

### Experimental Example 1

### Evaluation of Decellularized Composite Scaffold—DAPI (4′-6-diamidino-2-phenylindole) Staining and Immunofluorescent Staining

The following experiment was carried out to examine the decellularization of the composite scaffold.

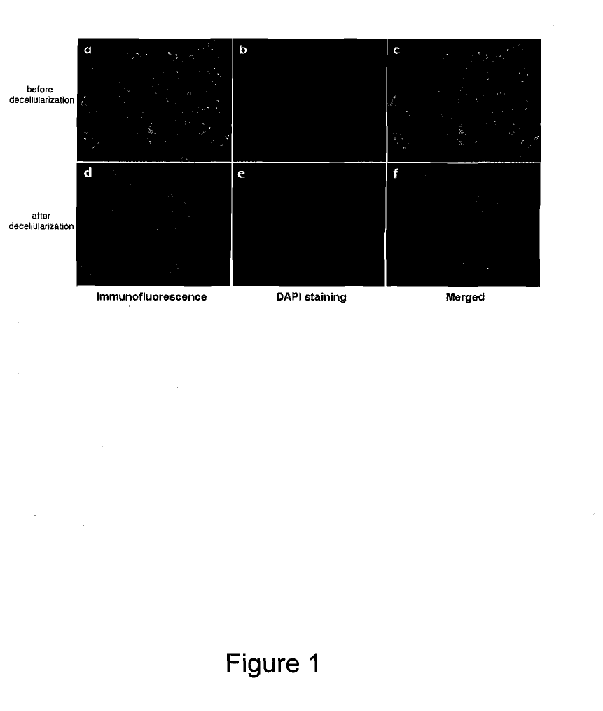

The sterilized composite scaffolds prepared in Example 1 were divided into three groups and transplanted with cells at a density of 5×10⁵ cells/scaffold [no cells transplanted (control), fibroblast NIH 3T3 transplanted, pre-osteoblast MC3T3-E1 transplanted], followed by incubation for one week at 37° C. in a 5% CO₂ atmosphere. After decellularization, each scaffold was washed with PBS and fixed for 20 min with 4% paraformaldehyde. Again, it was washed for 10 min with PBS containing 0.3% Triton X-100. An OCT (optimal cutting temperature) compound (a widely used embedding medium prior to frozen sectioning) was added and one hour after the scaffolds settled on the bottom they were cryopreserved with the OCT compound permeated thereinto. The scaffolds were then sectioned into slices of 10 μm in thickness using a microtome-cryostat. Immunofluorescent staining was conducted by reacting the slices for 10 min with 0.5% NaBH₄ (sodium borate or sodium borohydride)/PBS. The slices were washed three times with 0.1% TTBS (Tris-tween buffered saline) for 10 min each time and blocked for 1 hr with 2% BSA (bovine serum albumin)/TTBS. Overnight incubation at 4° C. with a primary antibody (1:40), washing three times with 0.1% TTBS for 10 minutes each time, incubation at room temperature for 1 hr with an FITC-secondary antibody (1:1000), and washing three times with 0.1% TTBS for 10 min each time were performed in that order. After being counterstained with DAPI, the slices were washed three times with 0.1% TTBS for 10 min each time to yield specimens. The presence of cell nuclei and the existence of type 1 collagen was examined under a fluorescence microscope. The results are shown in FIG. 1.

As seen in FIG. 1, DAPI staining revealed blue points in the scaffolds before the decellularization treatment while the blue stains disappeared from the scaffolds after the decellularization, demonstrating the removal of the nucleic part of the cells. In addition, green stains were observed in the scaffolds upon the immunofluorescent staining before as well as after the decellularization, indicating that type 1 collagen, representative of the ECM, remained even after decellularization.

### Experimental Example 2

### Capability of Decellularized Composite Scaffold to Induce Differentiation of Stem Cells: Alizarin Red Staining

The following experiment was conducted in order to examine whether the decellularized composite scaffold of the present invention was effective in differentiating stem cells into osteoblast-like cells.

Stem cells were transplanted in the decellularized composite scaffolds prepared in Examples 1 and 2 and cultured for two and four weeks in an osteogenic medium without BMP-2, respectively after which calcium distribution and intensity were analyzed by Alizarin red staining. The porous composite scaffold prepared in Example 1 was used as a control.

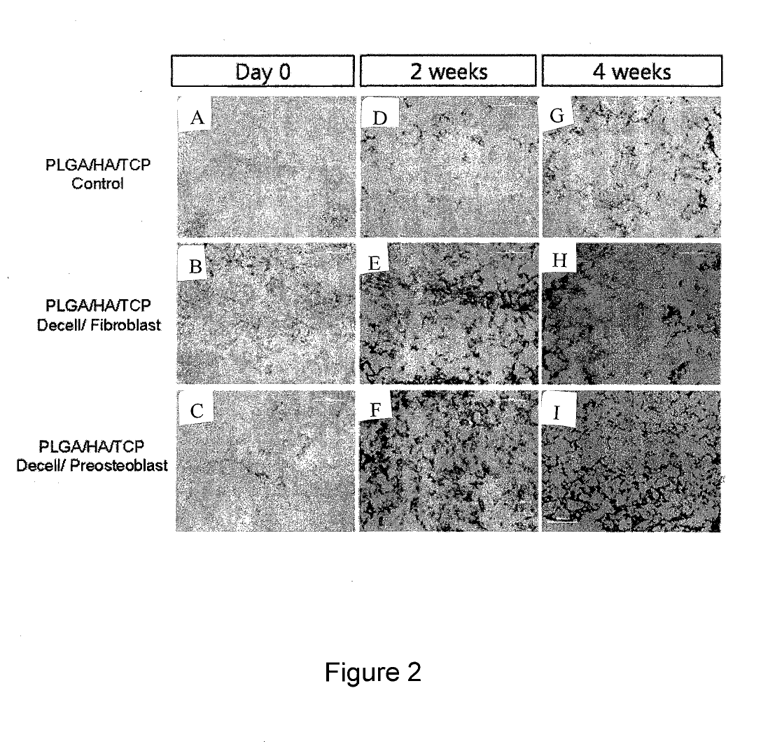

The results are shown in FIG. 2.

As seen in FIG. 2, red stains were observed in a greater area from the decellularized scaffold of the present invention than from the control, indicating that the decellularized scaffold of the present invention is much better in calcium distribution and deposition than the control and can provide an environment suitable for the differentiation of stem cells into osteoblast-like cells.

As described hitherto, the surface modification of a polymeric scaffold with an extracellular matrix secreted from a specific cell line allows cells to easily adhere to the scaffold and provides a biomimetic surface environment effective for the growth and differentiation of stem cells. Thus, the surface-modified polymeric scaffolds of the present invention can find a wide range of applications with stem cells, targeting specific tissue regeneration of a variety of different tissues including cartilage, bone, nerves, myocardium, liver, and skin.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

What is claimed:

1. A method for surface modification of a polymeric scaffold for stem cell transplantation, said method comprising the steps of:
a) preparing a porous composite scaffold by mixing a solution of an organic solvent containing a biodegradable organic polymer dissolved therein with an inorganic ingredient or a natural polymer;
b) foaming the mixture of step a) in the presence of NaHCO₃;
c) freeze-drying the mixture of step b) to make a porous composite scaffold;
d) subjecting the porous composite scaffold of step c) to plasma treatment and sterilization;
e) transplanting the scaffold with tissue derived-cells and culturing issue-derived cells thereon;
f) decellurizing the scaffold using a decellularization solution; and
g) transplanting and culturing stem cells in the decellularized scaffold.

2. The method of claim 1, wherein the biodegradable organic polymer is selected from the group consisting of polyglycolic acid (PGA), polylactic acid (PLA), poly(lactic-co-glycolic acid)(PLGA), poly-ε-caprolactone (PCL), polyamino acid, derivatives, and copolymers thereof.

3. The method of claim 1, wherein the inorganic ingredient is selected from the group consisting of hydroxyapatite (HA), β-tricalcium phosphate (β-TCP), biphasic calcium phosphate (BCP), calcium carbonate, and glass ceramics.

4. The method of claim 1, wherein the natural polymer is selected from the group consisting of collagen, a modified collagen, alginate, a modified alginate, hyaluronic acid, a modified hyaluronic acid, gelatin, a modified gelatin, chitosan, a modified chitosan, fibrin, and a modified fibrin.

5. The method of claim 1, wherein the organic solvent is selected from the group consisting of methylene chloride, hexane, chloroform, acetone, dioxane, tetrahydrofuran, and hexafluoroisopropane.

6. The method of claim 1, wherein the tissue-derived cells are selected from the group consisting of fibroblasts, chondrocytes, endothelial cells, smooth muscle cells, osteoblasts, hepatocytes, nerve cells, cardiomyocytes, intervertebral disc cells, and a combination thereof.

7. The method of claim 6, wherein the tissue-derived cells are autologous, homologous or heterologous cells.

8. The method of claim 1, wherein the decellularization solution is selected from the group consisting of a lysis solution, a hypotonic solution, a cationic-surfactant, an anionic-surfactant, a nonionic-surfactant, an RNase A, and a DNase I.

9. The method of claim 1, wherein the culturing of the tissue-derived cells or the stem cells is carried out in the presence of a biologically active biomolecule.

10. The method of claim 9, wherein the biologically active biomolecule is selected from the group consisting of transforming growth factor-beta (TGF-β), fibroblast growth factor (FGF), bone morphogenic protein (BMP), vascular endothelial growth factor (VEGF), epidermal growth factor (EGF), insulin-like growth factor (IGF), platelet-derived growth factor (PDGF), nerve growth factor (NGF), hepatocyte growth factor (HGF), placental growth factor (PIGF), granulocyte colony stimulating factor (G-CSF), medium supplements such as ascorbate 2-phosphate, and a combination thereof.

11. The method of claim 1, wherein the stem cells are autologous stem cells selected from the group consisting of embryonic stem cells, bone marrow-derived stem cells, adipose-derived stem cells, placenta-derived stem cells, induced pluripotent stem cells, and combinations thereof.
